(19) United States
(12) Patent Application Publication
(10) Pub. No.: US 2018/0099066 A1
(43) Pub. Date: Apr. 12, 2018

(54) Barrier Sleeve for Air Freshner for Controlled Release of Fragrance

(71) Applicants: Sven Dobler, Huntington, NY (US); Per Dobler, Huntington, NY (US); Valeriano Marinelli, Port Washington, NY (US)

(72) Inventors: Sven Dobler, Huntington, NY (US); Per Dobler, Huntington, NY (US); Valeriano Marinelli, Port Washington, NY (US)

(21) Appl. No.: 15/732,156

(22) Filed: Sep. 26, 2017

Related U.S. Application Data
(60) Provisional application No. 62/496,240, filed on Oct. 7, 2016.

(51) Int. Cl. A61L 9/12

(52) U.S. Cl. CPC A61L 9/127; A61L 2209/11

(57) ABSTRACT

An air freshener having a barrier for controlled release of fragrance is disclosed having a body having a top portion, a middle portion, and a bottom portion, an amount of fragrance loaded on the body, and a barrier positioned over a portion of the body.

### CROSS REFERENCE TO RELATED APPLICATION

This non provisional patent application claims priority to the provisional patent application having Ser. No. 62/496,240, filed on Oct. 7, 2016.

### FIELD OF THE DISCLOSURE

This disclosure generally relates to an air freshener and more particularly to an air freshener having a barrier sleeve for controlled release of fragrance.

### BACKGROUND

Air fresheners for automobiles typically consist of a printed paper card that is dosed with fragrance and provided with a string for hanging from a rear view mirror or other structure within the automobile or other vehicle. The air freshener is wrapped in an odor barrier containment package until it is to be used. The air freshener is also attached to a header card for displaying the air freshener on a peg at a retail location.

Most air fresheners work by dosing a liquid fragrance formulation into an absorbent material and then allowing the fragrance to be released during use by evaporation to the surrounding environment over time. The rate of evaporation is a critical factor of functionality. Quick evaporation of fragrance renders the air freshener overpowering upon removal of the wrapper and results in a very short usage period. To the consumer, this experience is unsatisfactory. The consumer may experience too much fragrance immediately which can be unpleasant and even irritating to the eyes and throat. Further, if the air freshener evaporates rapidly then the consumer will have to purchase a new air freshener in a few days since the air freshener has dried out and there is little if any fragrance left to be released. It is also possible that the fragrance will evaporate too slowly. If this occurs then there will be an insufficient impact to mask odors or even be noticed by the consumer. Since too little fragrance is evaporating on a daily basis there will be no initial impact by using the device and no sustained impact over time.

The rate of evaporation of the air freshener is a function of many interrelated factors. The thickness of the absorbent material used for the printed card or substrate is a factor. For example, the thinner the material that is used the faster the fragrance will be released. The thicker the material that is used the slower the fragrance will be released into the air. Another factor deals with the nature of the absorbent material or fragrance holder. The use of paper, plastics, gel, and wood are common, but liquid carriers such as pumps, bottles combined with wicks or paper, and liquid membrane containers are also used. The placement of the air freshener is a factor. In particular, the air freshener may be hung from a rear view mirror, be placed under a seat, positioned in an air vent, sprayed actively on demand or passively evaporated. The surface area of the air freshener also is a factor. The larger the surface area of the air freshener the quicker fragrance will be released into the air and the smaller the surface area of the air freshener the slower fragrance will be released. Another important design consideration is the fragrance formulation. A perfumer can increase or decrease the volatility of a fragrance formulation to control the rate of evaporation. The fragrance load can also be adjusted to control the rate of evaporation of fragrance. Specifically, the higher the fragrance load into the absorbent material or container the more power and longevity the air freshener can be expected to exhibit. The overall size and surface area of the air freshener is a factor that can be altered to control the rate of evaporation of fragrance. The smaller the carrier or surface area the less fragrance that the carrier can hold and release. The release of fragrance may be for a small period of time due to the size of the carrier and the impact of fragrance may be weak.

Conversely, a large surface area, will allow rapid and increased release of fragrance rendering the air freshener stronger. If the fragrance load is not also increased the air freshener will soon have lost its potency.

Ambient temperature also has an impact on the rate of evaporation. For example, the higher the ambient temperature the quicker the fragrance will evaporate. Also, the humidity of the environment in which the air freshener is placed must be considered. In dry air, the air freshener will release fragrance more rapidly then in humid air. Finally, the air flow surrounding the air freshener is another factor that impacts the rate of evaporation. The higher the air flow surrounding the air freshener the faster fragrance will be released.

Manufacturers and marketers of air fresheners have attempted to balance the above enumerated factors and constraints when designing air fresheners. In certain places and during certain climatic seasons the use of one type of air freshener may be preferred to that of another air freshener. When combined with consumer aesthetics and use preferences, a myriad of choices of air fresheners are available. For example, air fresheners such as paper, plastic, pumps, liquid membrane, vent clip auto, hanging, under seat, and table top are available for selection and purchase.

When designing air fresheners there are limitations in manufacturing thicknesses and characteristics of absorbent papers. Fourdrinier type paper machines can produce papers with smooth surfaces which is desirable for print decoration, but can generally produce a maximum thickness of 30 point. In order to increase the thickness it is necessary to use another paper ply that is glued to the 30 point thick ply to build up the thickness to an industry standard of 60 point. Multi-ply papers that use an adhesive are more prone to separation as the fragrance materials can dissolve the adhesive. Cylinder board machines can be used to produce thicker papers, but many of these papers are not suitable for printing and decorating due to their thickness and rougher surface properties. Plastics have limitations relative to thickness in the molding process as well.

The size and surface area for paper air fresheners is often constrained by three main factors. These three main factors are the width of a standard peg location at a retail location, and the concern to avoid obstruction of a driver's view, and the need for marketing and branding information as well as fragrance load. Generally, a paper air freshener has a thickness between 55 point and 80 point, but thinner and thicker air fresheners can also be found. The paper air freshener may have printing or decoration that is applied by use of lithographic offset or silk screen for thicker papers. The air freshener has a maximum size of generally 2.5 inches by 5 inches. The fragrance load will vary between 1 gram and 2 grams. However, the thicker the air freshener has problems such as being prone to separation and difficult to decorate. Also, increasing the size of the air freshener may prevent it from being displayed on a peg rack and decreasing the size will reduce the ability to load sufficient fragrance. In view of this, it should be appreciated that an air freshener manufacturer is faced with limitations in adjusting the various factors to create a long lasting air freshener having a steady, continuous, linear evaporation profile.

The air freshener having a barrier sleeve for controlled release of fragrance of the present disclosure is designed to obviate and overcome many of the disadvantages and shortcomings experienced with prior air fresheners. Moreover, the present disclosure is related to an air freshener having a barrier sleeve for controlled release of fragrance that is constructed so that fragrance is released primarily in the non-covered surface area of the air freshener and thus having a more linear release profile. Also, the sleeve may be shifted upon freshener so as to increase or decrease the amount of fragrance released.

### SUMMARY OF THE DISCLOSURE

In one form of the present disclosure an air freshener having a barrier sleeve for controlled release of fragrance is disclosed in which the air freshener comprises a body having a top portion, a middle portion, and a bottom portion, an amount of fragrance loaded on the air freshener, and a sleeve barrier positioned over the middle portion.

In another form of the present disclosure, an air freshener having a barrier for controlled release of fragrance is disclosed comprising a body having a top portion, a middle portion, and a bottom portion, an amount of fragrance loaded on the air freshener (“partial lamination”), and a patch positioned over the middle portion.

In yet another form of the present disclosure, an air freshener having a barrier for controlled release of fragrance is disclosed in which the air freshener comprises a body having a front side and a back side, a fragrance loaded on either the front side or the back side, or both sides, a second amount of fragrance loaded on the back side, a front lamination positioned on the entire surface of the front side, and a back lamination positioned on the entire surface of the back side.

In still another form of the present disclosure, an air freshener having a barrier for controlled release of fragrance is disclosed comprising a body having a front side and a back side, an amount of fragrance loaded either on the front side, back side, or both sides, and a lamination positioned on the entirety of only the front side.

In another form of the present disclosure, an air freshener having a barrier sleeve for controlled release of fragrance is disclosed in which the air freshener comprises a body having a front side and a back side, a first amount of fragrance loaded on the front side, a second amount of fragrance loaded on the back side, a front patch positioned on the front side, and a back patch positioned on the back side.

In light of the foregoing comments, it will be recognized that the present disclosure provides an air freshener having a barrier sleeve, patch or lamination for controlled release of fragrance that may be used for an extended period of time.

The property of the sleeve, patch, or lamination material is such that it retards or prevents the migration or evaporation to the air.

The present disclosure provides an air freshener having a barrier sleeve, patch or lamination for controlled release of fragrance that can be easily employed with highly reliable results and can be positioned in an automobile for an extended period of time without having to be replaced as readily.

The present disclosure also provides an air freshener having a barrier sleeve, patch or lamination for controlled release of fragrance that may be produced or manufactured on conventional equipment.

The present disclosure provides an air freshener having a barrier sleeve, patch or lamination for controlled release of fragrance that provides an initial release of fragrance that is not unpleasant or irritating.

The present disclosure provides an air freshener having a barrier sleeve, patch or lamination for controlled release of fragrance that is sized to fit on a conventional retail peg display.

These and other advantages of the present disclosure will become apparent to those skilled in the art after considering the following detailed specification in conjunction with the accompanying drawings.

### BRIEF DESCRIPTION OF THE DRAWINGS

FIG. 1 is a front view of an air freshener having a barrier sleeve for controlled release of fragrance constructed according to the present disclosure;

FIG. 2 is a back view of the air freshener having a barrier sleeve for controlled release of fragrance constructed according to the present disclosure;

FIG. 3 is a side view of the air freshener having a barrier sleeve for controlled release of fragrance shown in FIG. 1;

FIG. 4 is a front perspective view of another embodiment of an air freshener having a barrier patch for controlled release of fragrance constructed according to the present disclosure;

FIG. 5 is side view of the air freshener having a barrier patch for controlled release of fragrance shown in FIG. 4;

FIG. 6 is is a side view of another embodiment of an air freshener having a full barrier lamination on the front of the air freshener for controlled release of fragrance constructed according to the present disclosure;

FIG. 7 is a side view of another embodiment of an air freshener having a full barrier lamination on both the front and back sides of the air freshener for controlled release of fragrance constructed according to the present disclosure;

FIG. 8 is a side view of another embodiment of an air freshener having a barrier patch on the front and back sides of the air freshener for controlled release of fragrance constructed according to the present disclosure;

FIG. 9 is a graph of rapid evaporation of fragrance from an air freshener;

FIG. 10 is a graph of slow evaporation of fragrance from an air freshener; and

FIG. 11 is a graph of controlled linear release of fragrance from an air freshener having a barrier sleeve, patch or full lamination for controlled release of fragrance constructed according to the present disclosure.

### DETAILED DESCRIPTION OF PREFERRED EMBODIMENTS

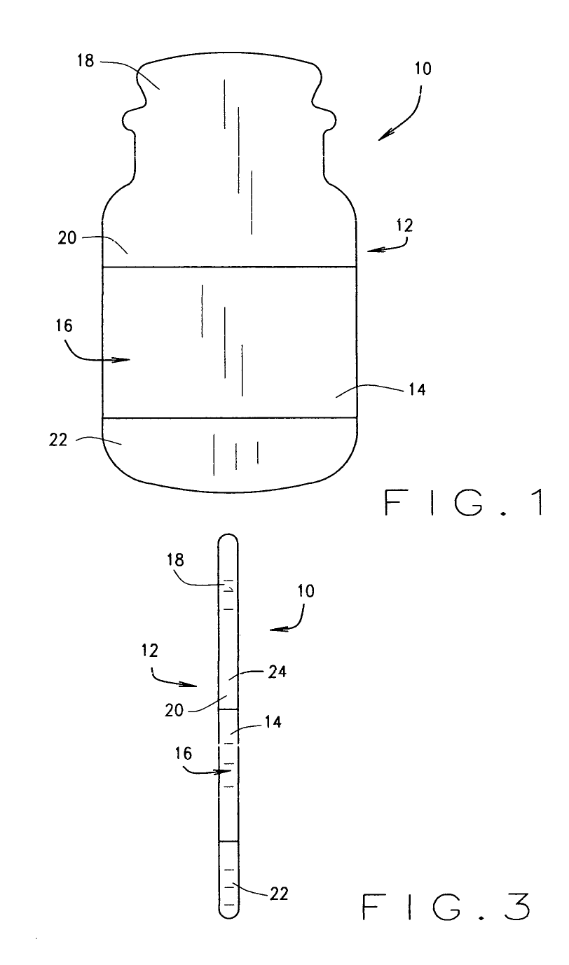

Referring now to the drawings, wherein like numbers refer to like items, number 10 identifies a preferred embodiment of an air freshener having a barrier sleeve for controlled release of fragrance constructed according to the present disclosure. With reference now to FIG. 1, the air freshener 10 comprises a body 12, a fragrance 14 deposited somewhere on air freshener 10, and a barrier sleeve or band 16 positioned over the and around the air freshener 10. The body 12 has a top portion 18, a middle portion 20, and a bottom portion 22. The barrier sleeve 16 is shown being positioned over the middle portion 20. Although not shown, after the air freshener 10 is constructed, the air freshener 10 may be placed in a package for shipping and displaying on a retail peg display. The sleeve 16 is positioned over and around the air freshener 10 after the fragrance 14 has been loaded on the body 12. The sleeve 16 may be made with an odor barrier or semi-odor film material such as polyvinylchloride (PVC), polyester, polyethylene (LDPE and HDPE), polyethylene terephthalate, acetate, ethyl vinyl alcohol, polyamide (nylon), methyl-ethyl-cellulose, styrene, polystyrene, polypropylene, single and multi-layer films of these materials, together with foils, and other polymer and co-polymer materials. This listing of materials is for purposes of example only and not in a limiting manner.

The sleeve 16 is sized and shaped to fit around the middle portion 20 of the air freshener 10. The sleeve 16 remains attached to the body 12 of the air freshener 10 during use. The sleeve 16 may be attached to the body 12 by friction, adhesion, cohesion, or shrink sealing under heat or ultraviolet light exposure. The size of the sleeve 16 may be adjusted to increase or decrease the surface area of the body 12 that is exposed to the air. The choice of material that the sleeve 16 is constructed of may be chosen based on the nature and extent of its odor barrier properties and the level of containment desired. After the sleeve 16 is attached to the body 12, the air freshener 10 is packaged typically in an odor barrier packaging to preserve the air freshener 10 until the air freshener 10 is to be used. The odor barrier packaging is then typically attached to a header card that is used in conjunction with a retail peg display to display the air freshener 10 for purchase at a retail establishment. The sleeve 16 is to remain on the air freshener 10 when in use to allow the fragrance 14 to wick to the exposed portions, such as the top portion 18 and the bottom portion 22 of the body 12. The sleeve 16 reduces the surface area of the body 12 through which the fragrance 14 can evaporate into the air or atmosphere. With use of the sleeve 16, a significant amount of fragrance 14 can be loaded into the absorbent material of the body 12. The fragrance 14 will be released through the controlled exposed surface area of the portions 18 and 22 and through the controlled odor barrier sleeve 16. The air freshener 10 will have increased longevity, will reduce the amount of fragrance 14 initially released, and will provide a more linear release profile. Although the sleeve 16 is shown covering the middle portion 20, it is contemplated that the sleeve 16 may be wider in that the sleeve 16 can also cover portions of the top portion 18 and the bottom portion 22. It is further possible that the sleeve 16 may be narrower than depicted so that the sleeve 16 covers only a portion of the middle portion 20. As can be appreciated, the width and position of the sleeve 16 may be adjusted as required to control the release of the fragrance 14 from the body 12.

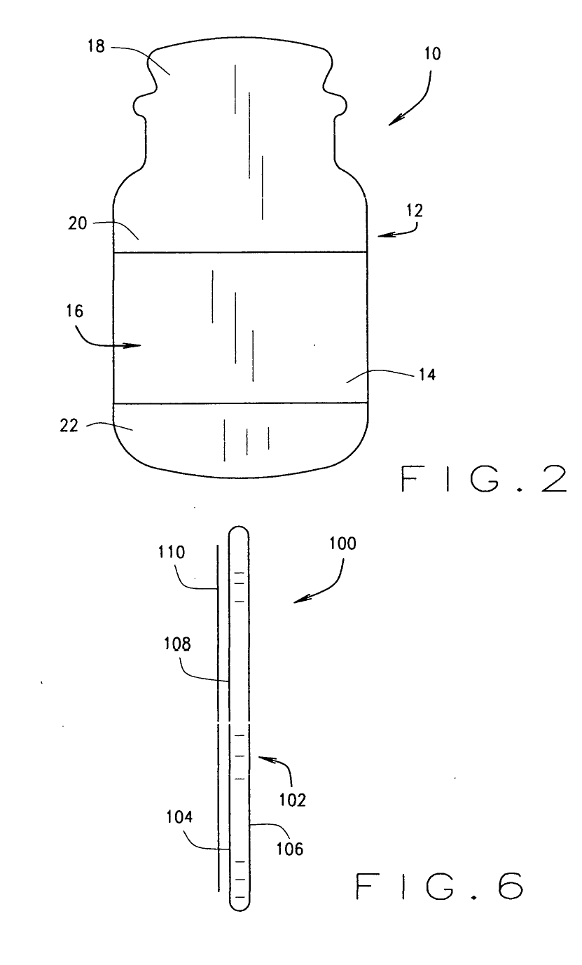

FIG. 2 illustrates a back view of the air freshener 10. The air freshener 10 comprises the body 12, the fragrance 14, and the sleeve 16 that has been wrapped around the body 12. The body 12 is also shown to have the top portion 18, the middle portion 20, and the bottom portion 22. The fragrance 14 is capable of wicking in a controlled manner from the sleeve 16 to the top portion 18 and the bottom portion 22 during use.

Referring now in particular to FIG. 3, a side view of the air freshener 10 is depicted. The air freshener 10 has the sleeve 16 wrapped around a side 24 of the body 12. The body 12 also has the fragrance 14 loaded on to any portion of the air freshener 10. The fragrance 14 is capable of wicking to the top portion 18 and the bottom portion 22.

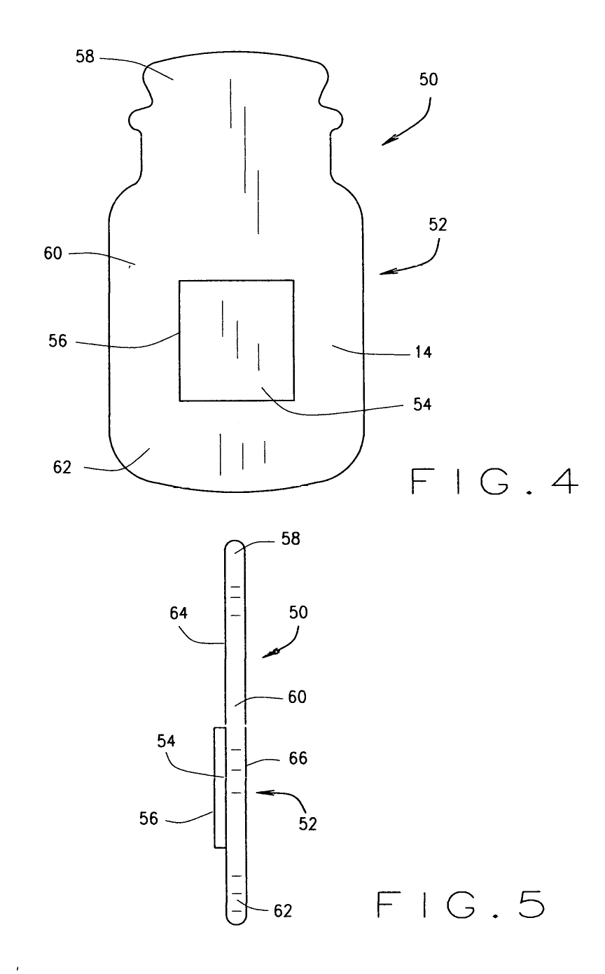

With reference now to FIG. 4, another embodiment of an air freshener having a barrier patch for controlled release of fragrance 50 is shown. The air freshener 50 comprises comprises a body 52, a fragrance 54, and a barrier patch 56 positioned on the air freshener 50. The body 52 has a top portion 58, a middle portion 60, and a bottom portion 62. The barrier patch 56 is shown being positioned over the middle portion 60. The patch 56 is positioned in the middle portion 60 after the fragrance 54 has been loaded on the body 52. The patch 56 may be made with any of the previously discussed and disclosed odor barrier or semi-odor film materials, and additionally any coating that provides odor barrier or retention qualities to cover only a portion of the truck body 52. The sleeve and the patch structurally are different, but they serve the same purpose.

The patch 56 is sized and shaped to cover only a portion of the front body 56. The patch 56 remains attached to the body 52 of the air freshener 50 during use. The patch 56 may be attached to the body 52 by friction, adhesion, cohesion, or shrink sealing under heat or ultraviolet exposure or if a coating may be applied by lithographic offset, flexography, silkscreen, or extrusion. The size of the patch 56 may be adjusted to increase or decrease the surface area of the body 52 that is exposed to the air. The choice of material that the sleeve 56 is constructed of may be chosen based on the nature and extent of its odor barrier properties and the level of containment desired. After the sleeve 56 is attached to the body 52, the air freshener 50 is typically packaged in an odor barrier packaging to preserve the air freshener 50 until the air freshener 50 is to be used. The odor barrier packaging is then typically attached to a header card that is used in conjunction with a retail peg display to display the air freshener 50 for purchase at a retail establishment. With the patch 56 in place, the fragrance 54 is allowed to wick to the exposed portions, such as the top portion 58, the middle portion 60, and the bottom portion 62 of the body 52. The patch 56 reduces the open surface area of the body 52 through which the fragrance 54 can evaporate into the air or atmosphere. With use of the patch 56, a significant amount of fragrance 54 can be loaded into the absorbent material of the body 52. The fragrance 54 will be released through the controlled exposed surface area of the portions 58, 60, and 62 and through the controlled odor barrier patch 56. The air freshener 50 will have increased longevity, will reduce the amount of fragrance 54 initially released, and will provide a more linear release profile.

FIG. 5 depicts a side view of the air freshener 50. The body 52 has a front side 64 having the top portion 58, the middle portion 60, and the bottom portion 62. The fragrance 54 has been dosed anywhere on the front 64 or back 66 of the air freshener. The patch 56 has been placed over a limited area in portions 58, 60, or 62 of side 64 of body 52. As can be appreciated, the air freshener 50 may be placed in an odor barrier packaging that is attached to a header card for displaying the air freshener 50 in a retail store.

Referring now to FIG. 6, another embodiment of an air freshener having a barrier lamination for controlled release of fragrance 100 is illustrated. The air freshener 100 comprises a body 102 having a front side 104 and a back side 106. The front side 104 has an amount of fragrance 108 loaded therein or thereon and a film barrier lamination 110 over the front side 104. The lamination 110 may consists of any of the previously discussed and disclosed odor barrier or semi-odor film materials. The lamination 110 or coatings may be laminated on the body 102 before or after the fragrance 108 is loaded. The lamination 110 serves the same purpose as the sleeve 16 or the patch 56 in that the lamination 110 reduces the surface area of the air freshener 100 through which the fragrance 108 can readily evaporate and be released into the air. As well as coatings, the lamination 110 may be placed on the body 102 by mechanical attachment such as stapling, grommeting, folding, or punching or by other means such as adhesion or cohesion, and coatings by lithographic offset flexography, silkscreen, or extrusion.

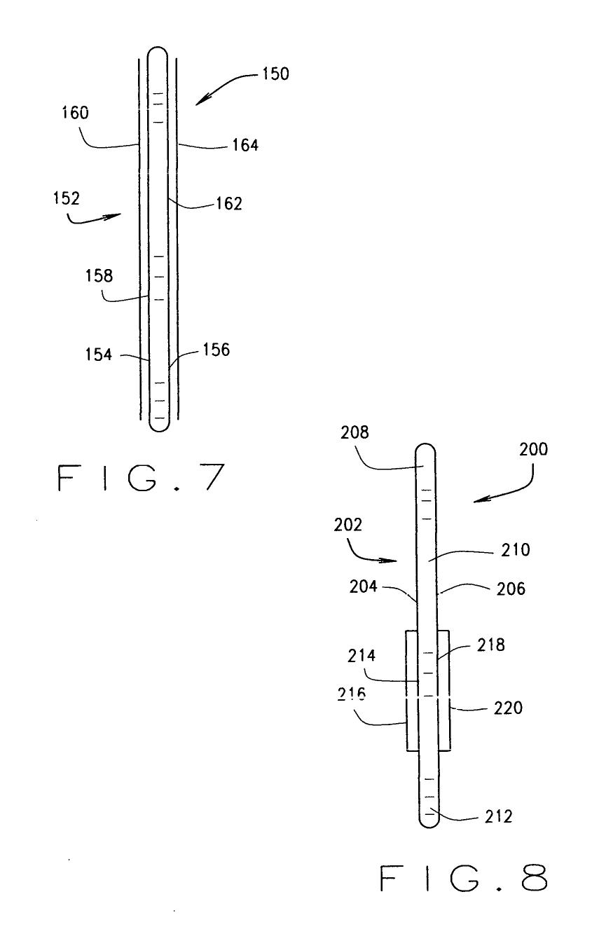

FIG. 7 shows another embodiment of an air freshener having a barrier lamination for controlled release of fragrance 150 constructed according to the present disclosure. The air freshener 150 is shown comprising a body 152 having a front side 154 and a back side 156. The front side 154 has an amount of fragrance 158 loaded therein or thereon and a front film barrier lamination 160 over the front side 154. The back side 156 also has an amount of fragrance 162 loaded therein or thereon and a back film barrier lamination 164 over the back side 156. The front lamination 160 and the back lamination 164 may each consist of any of the previously discussed and disclosed odor barrier or semi-odor film materials or coatings. The laminations 160 and 164 may be laminated on the body 152 before or after the fragrances 158 and 162 are loaded. The laminations 160 and 164 each serve to reduce or restrict the surface area of the air freshener 150 through which the fragrances 158 and 162 can readily evaporate and be released into the atmosphere. The laminations 160 and 164 may be placed on the body 152 by mechanical attachment such as stapling, grommeting, folding, or punching or by other means such as adhesion or cohesion and the coatings may be applied by lithographic offset flexography, silkscreen, or extrusion.

Referring now in particular to FIG. 8, another embodiment of an air freshener having two barrier patches for controlled release of fragrance 200 constructed according to the present disclosure is illustrated. The air freshener 200 comprises a body 202 having a front side 204, a back side 206, a top portion 208, a middle portion 210, and a bottom portion 212. The front side 204 has an amount of fragrance 214 loaded therein or thereon and a front patch 216 positioned over a limited areas of front side 204. The back side 206 also has an amount of fragrance 218 loaded therein or thereon and a back patch 220 positioned over a limited area of back side 206. The front patch 216 and the back patch 220 may each consist of any of the previously discussed and disclosed odor barrier or semi-odor film materials or coatings. The patches 216 and 220 are sized and shaped to cover some of the fragrances 214 and 218, respectively. As can be appreciated, the fragrance 214 is allowed to wick into the top portion 208, the middle portion 210, and the bottom portion 212 on the front side 204 and the fragrance 218 is allowed to wick into the top portion 208, the middle portion 210, and the bottom portion 212 on the back side 206 in a controlled manner.

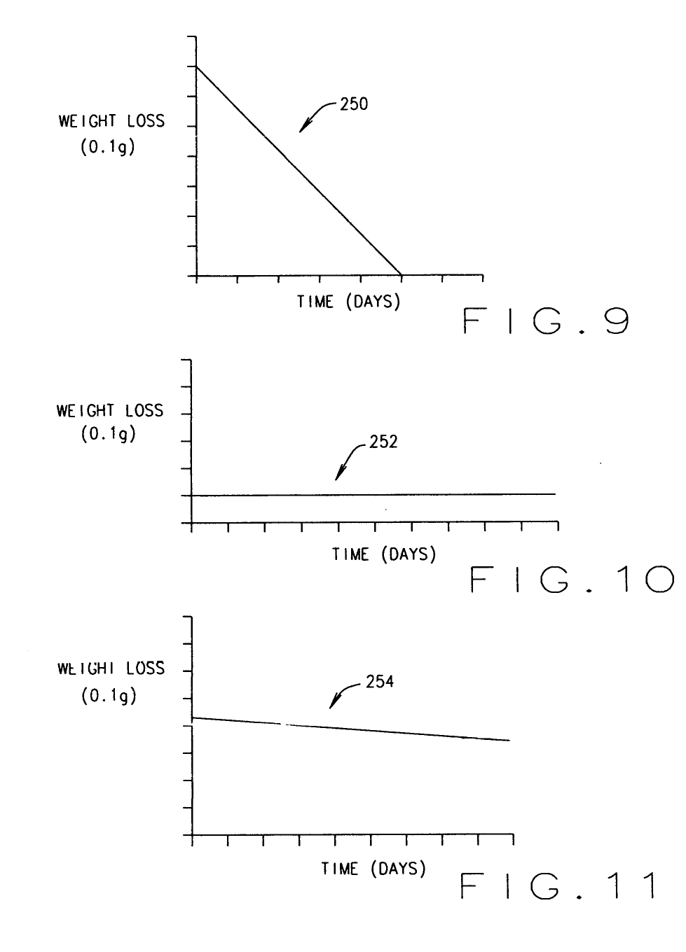

FIG. 9 is a graph 250 showing rapid evaporation of fragrance from a prior air freshener. The graph 250 indicates that there is an initial impact of fragrance and then a short life for the air freshener.

FIG. 10 is a graph 252 showing slow evaporation of fragrance from a prior air freshener. In this case, the graph 252 illustrates that there is little if any impact of fragrance provided by the air freshener and the air freshener may last for a long period of time with little function.

A graph 254 is depicted in FIG. 11 of the evaporation of the air freshener 10. The graph 254 indicates that there is steady evaporation of the fragrance 14 with good initial impact and an acceptable service life of the air freshener 10. As should be appreciated, the air freshener 10 having these characteristics is preferred over the air fresheners that form the basis for the graphs 250 and 252.

From all that has been said, it will be clear that there has thus been shown and described herein an air freshener having a barrier sleeve for controlled release of fragrance. It will become apparent to those skilled in the art, however, that many changes, modifications, variations, and other uses and applications of the subject air freshener having a barrier sleeve for controlled release of fragrance are possible and contemplated. All changes, modifications, variations, and other uses and applications which do not depart from the spirit and scope of the disclosure are deemed to be covered by the disclosure, which is limited only by the claims which follow.

What is claimed is:

1. An air freshener having a barrier sleeve for controlled release of fragrance comprising:
a body having a top portion, a middle portion, and a bottom portion;
an amount of fragrance loaded on the body portion; and
a sleeve barrier positioned over the middle portion.

2. The air freshener having a barrier sleeve for controlled release of fragrance of claim 1 wherein the sleeve comprises an odor barrier film or odor barrier coating.

3. The air freshener having a barrier sleeve for controlled release of fragrance of claim 1 wherein the sleeve is attached to the body by friction, adhesion, cohesion, or shrink sealing under heat or ultraviolet exposure.

4. The air freshener having a barrier coating applied by conventional coating equipment, such as lithographic offset, flexography, silkscreen, or extrusion.

5. The air freshener having a barrier sleeve for controlled release of fragrance of claim 1 wherein the sleeve comprises polyvinylchloride (PVC), polyester, polyethylene (LDPE and HDPE), polyethylene terephthalate, acetate, ethyl vinyl alcohol, polyamide (nylon), methyl-ethyl-cellulose, styrene, polystyrene, polypropylene, single and multi-layer films of these materials, together with foils, and other polymer and co-polymer materials.

6. The air freshener having a barrier sleeve for controlled release of fragrance of claim 1 wherein the sleeve is sized and shaped to cover the middle portion of the body.

7. The air freshener having a barrier sleeve for controlled release of fragrance of claim 1 wherein the fragrance wicks from the middle portion to the top portion and the bottom portion.

8. An air freshener having a barrier sleeve for controlled release of fragrance comprising:
a body having a top portion, a middle portion, and a bottom portion;
an amount of fragrance loaded on the body portion; and
a patch positioned over a limited surface of any portion.

9. An air freshener having a barrier sleeve for controlled release of fragrance comprising:
a body having a front side and a back side;
a first amount of fragrance loaded on the body;
a front lamination positioned on the front side; and
a back lamination positioned on the back side.

10. An air freshener having a barrier sleeve for controlled release of fragrance comprising:
a body having a front side and a back side;
an amount of fragrance loaded on the body; and
a lamination positioned on the front side.

11. An air freshener having a barrier sleeve for controlled release of fragrance comprising:
a body having a front side and a back side;
an amount of fragrance loaded on the body;
a second amount of fragrance loaded on the back side;
a front patch positioned on a limited surface area of the front side; and
a back patch positioned on a limited surface area of the back side.
